(19) United States
(12) Patent Application Publication
(10) Pub. No.: US 2009/0181952 A1
(43) Pub. Date: Jul. 16, 2009

(54) COMPOUNDS USEFUL AS ALPHA7 NICOTINIC ACETYLCHOLINE RECEPTOR AGONISTS

(75) Inventors: Simon N. Haydar, Newtown, PA (US); Riccardo Zanaletti, Colle Val d'Elsa (IT)

Correspondence Address: CHOATE, HALL & STEWART LLP/WYETH, PATENT GROUP, TWO INTERNATIONAL PLACE, BOSTON, MA, 02110, US

(73) Assignees: Wyeth, Madison, NJ (US); Siena Biotech S.p.A., Siena (IT)

(21) Appl. No.: 12/353,804

(22) Filed: Jan. 14, 2009

Related U.S. Application Data
(60) Provisional application No. 61/021,017, filed on Jan. 14, 2008.

(51) Int. Cl. A61K 31/5513; C07D 403/12

(52) U.S. Cl. 514/218; 540/575

(57) ABSTRACT

The present invention provides compounds and compositions, and methods of using them to modulate α7 nicotinic acetylcholine receptors and/or to treat any of a variety of disorders, diseases, and conditions. Provided compounds can affect, among other things, neurological, psychiatric and/or inflammatory systems.

### CROSS REFERENCE TO RELATED APPLICATIONS

This application claims priority to U.S. provisional patent application Ser. No. 61/021,017, filed Jan. 14, 2008, the entirety of which is hereby incorporated herein by reference.

### FIELD OF THE INVENTION

The present invention relates to compounds with α7 nicotinic acetylcholine receptor (α7 nAChR) agonistic activity, pharmaceutical compositions, and the use thereof for the treatment of neurological, psychiatric, and inflammatory diseases.

### BACKGROUND OF THE INVENTION

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including for example, schizophrenia, anxiety, mania, depression, manic depression, Tourette's syndrome, Parkinson's disease, Huntington's disease, cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), and other uses such as treatment of nicotine addiction, inducing smoking cessation, treating pain (e.g. analgesic use), providing neuroprotection, and treating jetlag. See for example WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Different heterocyclic compounds carrying a basic nitrogen and exhibiting nicotinic and muscarinic acetylcholine receptor affinity or claimed for use in Alzheimer disease have been described, e.g. 1H-pyrazole and pyrrole-azabicyclic compounds (WO2004013137); nicotinic acetylcholine agonists (WO2004039366); ureido-pyrazole derivatives (WO0112188); oxadiazole derivatives having acetylcholinesterase-inhibitory activity and muscarinic agonist activity (WO9313083); pyrazole-3-carboxylic acid amide derivatives as pharmaceutical compounds (WO2006077428); arylpiperidines (WO2004006924); ureidoalkylpiperidines (U.S. Pat. No. 6,605,623); compounds with activity on muscarinic receptors (WO9950247). In addition, modulators of alpha7 nicotinic acetylcholine receptor are disclosed in WO06008133, in the name of the same applicant.

### SUMMARY

Among other things, the invention provides novel compounds acting as full or partial agonists at the α7 nicotinic acetylcholine receptor (α7 nAChR), pharmaceutical compositions, and the use thereof for the treatment of diseases that may benefit from the activation of the alpha 7 nicotinic acetylcholine receptor such as neurological, neurodegenerative, psychiatric, cognitive, immunological, inflammatory, metabolic, addiction, nociceptive, and sexual disorders, in particular Alzheimer's disease, schizophrenia, and/or others.

### DESCRIPTION OF THE DRAWING

FIG. 1 shows extracted ion chromatograms of compound I-6 and its metabolites in rat and dog plasma

FIG. 2 shows an ion chromatograms of compound I-6 and its metabolites in rat plasma.

FIG. 3 shows HPLC chromatograms of selected compounds described in Example 3.

### DESCRIPTION OF CERTAIN PARTICULAR EMBODIMENTS

### Compounds

The present invention provides compound useful as agonists of the α7 nicotinic acetylcholine receptor (α7 nAChR).

In certain embodiments, the present invention provides a compound of formula I:

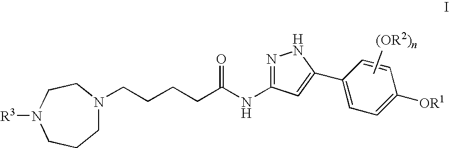

or a pharmaceutically acceptable salt thereof, wherein:
n is 0-4;
R¹ is hydrogen, methyl, or —SO₃H;
each R² is independently hydrogen or —SO₃H; and
R³ is hydrogen or —C(O)Me.

In certain embodiments, the n group of formula I is 0. In other embodiments, the n group of formula I is 1-4. In some embodiments, the n group of formula I is 1.

In certain embodiments, the R¹ group of formula I is hydrogen.

In some embodiments, the R¹ group of formula I is —SO₃H.

In certain embodiments, each R² group of formula I is hydrogen.

In certain embodiments, the R³ group of formula I is hydrogen. In certain embodiments, the R³ group of formula I is —C(O)Me. In some embodiments, when R¹ is methyl and R³ is —C(O)Me, n is not zero.

Exemplary compounds of formula I are set forth in Table 1, below.

TABLE 1
Exemplary Compounds of Formula I:

| | |
|---|---|
| | I-1 |
| 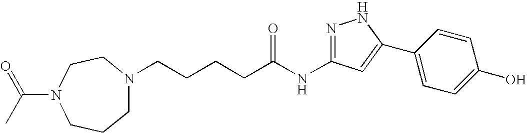 | |
| 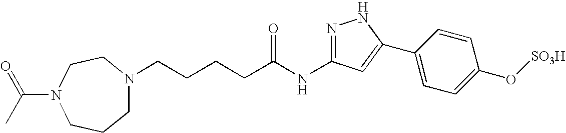 | I-2 |
| 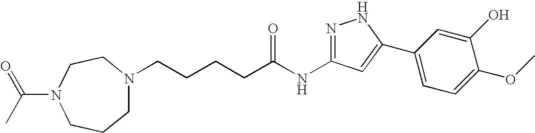 | I-3 |
| 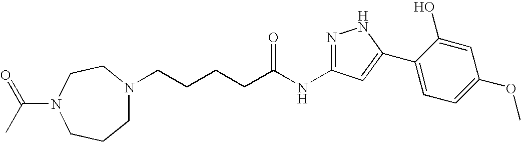 | I-4 |
| 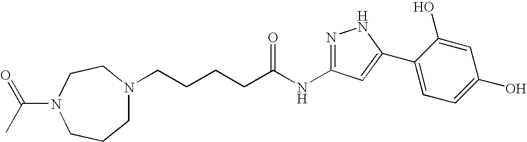 | I-5 |
| 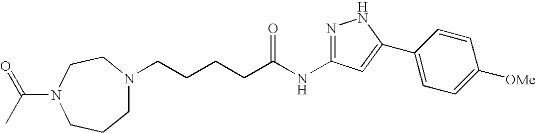 | I-6 |
| 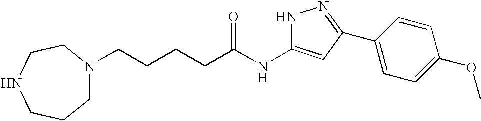 | I-7 |

Compounds of the present invention are also useful for the study of full or partial activation at the α7 nicotinic acetylcholine receptor (α7 nAChR) in biological and pathological phenomena and the comparative evaluation of α7 nAChR agonists or partial agonists.

The present compounds were discovered as a result of metabolic studies of α7 nAChR agonists or partial agonists. Without wishing to be bound by theory, it is believed that the present compounds are metabolites of α7 nAChR agonists or partial agonists. Accordingly, the present compounds are also useful for studying the effects of such α7 nAChR agonists or partial agonists in vivo or in vitro.

As will be readily apparent to one skilled in the art, the unsubstituted ring nitrogen pyrazoles and imidazoles, as in the compounds of the present invention, are known to rapidly equilibrate in solution, as mixtures of both tautomers:

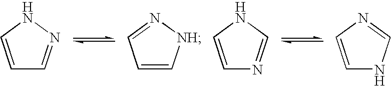

in the following description therefore, where only one tautomer is indicated for compounds of Formula (I), the other tautomer is also intended as within the scope of the present invention.

Compounds of the invention can be in the form of free bases or acid addition salts, preferably salts with pharmaceutically acceptable acids. The invention also provides separated isomers and diastereoisomers of compounds of Formula (I), or mixtures thereof (e.g. racemic and diastereomeric mixtures), as well as isotopic compositions.

Pharmacological activity of a representative group of compounds of Formula (I) was demonstrated in an in vitro assay utilising cells stably transfected with the alpha 7 nicotinic acetylcholine receptor and cells expressing the alpha 1 and alpha 3 nicotinic acetylcholine receptors and 5HT₃ receptor as controls for selectivity.

Compounds of Formula (I) may be provided according to the present invention in any of a variety of useful forms, for example as pharmaceutically acceptable salts, as particular crystal forms, etc. In some embodiments, prodrugs of one or more compounds of Formula (I) are provided. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

### Uses

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including, for example, schizophrenia, anxiety, mania, depression, manic depression, Tourette's syndrome, Parkinson's disease, Huntington's disease, cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder,), and other uses such as treatment of nicotine addiction, inducing smoking cessation, treating pain (i.e., analgesic use), providing neuroprotection, and treating jetlag. See, e.g., WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Thus, in accordance with the invention, there is provided a method of treating a patient, especially a human, suffering from any of psychotic diseases, neurodegenerative diseases involving a dysfunction of the cholinergic system, and/or conditions of memory and/or cognition impairment, including, for example, schizophrenia, anxiety, mania, depression, manic depression, Tourette's syndrome, Parkinson's disease, Huntington's disease, and/or cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder) comprising administering to the patient an effective amount of a compound according to Formula (I).

Neurodegenerative disorders whose treatment is included within the methods of the present invention include, but are not limited to, treatment and/or prophylaxis of Alzheimer's diseases, Pick's disease (Friedland, Dementia, (1993) 192-203; Procter, Dement Geriatr Cogn Disord. (1999) 80-4; Sparks, Arch Neurol. (1991) 796-9; Mizukami, Acta Neuropathol. (1989) 52-6; Hansen, Am J Pathol. (1988) 507-18), diffuse Lewy Body disease, progressive supranuclear palsy (Steel-Richardson syndrome, see Whitehouse, J Neural Transm Suppl. (1987) 24:175-82; Whitehouse, Arch Neurol. (1988) 45(7):722-4; Whitehouse, Alzheimer Dis Assoc Disord. 1995; 9 Suppl 2:3-5; Warren, Brain. 2005 February; 128(Pt 2):239-49), multisystem degeneration (Shy-Drager syndrome), motor neuron diseases including amyotrophic lateral sclerosis (Nakamizo, Biochem Biophys Res Commun. (2005) 330(4), 1285-9; Messi, FEBS Lett. (1997) 411(1):32-8; Mohammadi, Muscle Nerve. (2002) October; 26(4):539-45; Hanagasi, Brain Res Cogn Brain Res. (2002) 14(2):234-44; Crochemore, Neurochem Int. (2005) 46(5):357-68), degenerative ataxias, cortical basal degeneration, ALS-Parkinson's-Dementia complex of Guam, subacute sclerosing panencephalitis, Huntington's disease (Kanazawa, J Neurol Sci. (1985) 151-65; Manyam, J Neurol. (1990) 281-4; Lange, J Neurol. (1992) 103-4; Vetter, J. Neurochem. (2003) 1054-63; De Tommaso, Mov Disord. (2004) 1516-8; Smith, Hum Mol. Genet. (2006) 3119-31; Cubo, Neurology. (2006) 1268-71), Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, prion diseases (such as Creutzfeldt-Jakob, Gerstmann-Straussler-Scheinker disease, Kuru and fatal familial insomnia), and neurodegenerative disorders resulting from cerebral ischemia or infarction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

In addition, α7nACh receptor agonists, such as the compounds of the present invention can be used to treat age-related dementia and other dementias and conditions with memory loss including age-related memory loss, senility, vascular dementia, diffuse white matter disease (Binswanger's disease), dementia of endocrine or metabolic origin, dementia of head trauma and diffuse brain damage, dementia pugilistica, alcoholism related dementia (Korsakoff Syndrome) and frontal lobe dementia. See, e.g., WO 99/62505., Tomimoto Dement Geriatr Cogn Disord. (2005), 282-8; Tohgi—J Neural Transm. (1996), 1211-20; Casamenti, Neuroscience (1993) 465-71, Kopelman, Br J Psychiatry (1995) 154-73; Cochrane, Alcohol Alcohol. (2005) 151-4).

Amyloid precursor protein (APP) and Aβ peptides derived therefrom, e.g., Aβ1-42 and other fragments, are known to be involved in the pathology of Alzheimer's disease. The Aβ1-42 peptides are not only implicated in neurotoxicity but also are known to inhibit cholinergic transmitter function. Further, it has been determined that Aβ peptides bind to α7nACh receptors. The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. See for example Czura, C J et al., J. Intern. Med., (2005) 257(2), 156-66; Wang, H. et al Nature (2003) 421: 384-388; de Jonge British Journal of Pharmacology (2007) 151, 915-929. The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol. Reproduct. 68: 1348-1353, 2003). In addition, nicotinic receptors have been implicated as playing a role in the body's response to alcohol ingestion. α7nACh receptor agonists such as compounds provided herein, therefore, are also used in the treatment of these disorders, diseases, and conditions.

For example, agonists for the α7nACh receptor subtypes can also be used in the treatment of nicotine addiction, inducing smoking cessation, treating pain, and treating jetlag, obesity, diabetes, sexual and fertility disorders (eg. Premature ejaculation or vaginal dryness, see U.S. Pat. No. 6,448,276), drug abuse (Solinas, Journal of Neuroscience (2007) 27(21), 5615-5620), and inflammation (Wang H, et al. (2003) Nature 421:384-388).

A number of recent observations point to a potential neuroprotective effect of nicotine in a variety of neurodegeneration models in animals and in cultured cells, involving excitotoxic insults (Prendergast, M. A., et al. Med. Sci. Monit. (2001), 7, 1153-1160; Gamido, R., et al. (2001), J. Neurochem. 76, 1395-1403; Semba, J., et al. (1996) Brain Res. 735, 335-338; Shimohama, S., et al. (1996), Ann. N.Y. Acad. Sci. 777, 356-361; Akaike, A., et al. (1994) Brain Res. 644, 181-187), trophic deprivation (Yamashita, H., Nakamura, S. (1996) Neurosci. Lett. 213, 145-147), ischemia (Shimohama, S. (1998) Brain Res. 779, 359-363), trauma (Socci, D. J., Arendash, G. W. (1996) Mol. Chem. Neuropathol. 27, 285-305), Aβ-mediated neuronal death (Rusted, J. M., et al. (2000) Behav. Brain Res. 113, 121-129; Kihara, T., et al. (1997) Ann. Neurol. 42, 159-163; Kihara, T., et al. (2001) J. Biol. Chem. 276, 13541-13546) and protein-aggregation mediated neuronal degeneration (Kelton, M. C. et al. (2000) Brain Cogn 43, 274-282). In many instances where nicotine displays a neuroprotective effect, a direct involvement of receptors comprising the α7 subtype has been invoked (Shimohama, S. et al. (1998) Brain Res. 779, 359-363; Kihara, T., et al. (2001) J. Biol. Chem. 276, 13541-13546; Kelton, M. C., et al. (2000) Brain Cogn 43, 274-282; Kem, W. R. (2000) Behav. Brain Res. 113, 169-181; Dajas-Bailador, F. A., et al. (2000) Neuropharmacology 39, 2799-2807; Strahlendorf, J. C., et al. (2001) Brain Res. 901, 71-78) suggesting that activation of α7 subtype-containing nicotinic acetylcholine receptors may be instrumental in mediating the neuroprotective effects of nicotine. Available data suggest that the α7 nicotinic acetylcholine receptor represents a valid molecular target for the development of agonists/positive modulators active as neuroprotective molecules. Indeed, α7 nicotinic receptor agonists have already been identified and evaluated as possible leads for the development of neuroprotective drugs (Jonnala, R. R., et al. (2002) Life Sci. 70, 1543-1554; Bencherif, M., et al. (2000) Eur. J. Pharmacol. 409, 45-55; Donnelly-Roberts, D. L., et al. (1996) Brain Res. 719, 36-44; Meyer, E. M., et al. (1998) J. Pharmacol. Exp. Ther. 284, 1026-1032; Stevens, T. R., et al. (2003) J. Neuroscience 23, 10093-10099). Compounds described herein can be used to treat such diseases.

In accordance with the invention, there is provided a method of treating a patient, especially a human, suffering from age-related dementia and other dementias and conditions with memory loss comprising administering to the patient an effective amount of a compound according to Formula (I).

The present invention includes methods of treating patients suffering from memory impairment due to, for example, mild cognitive impairment due to aging, Alzheimer's disease, schizophrenia, Parkinson's disease, Huntington's disease, Pick's disease, Creutzfeldt-Jakob disease, depression, aging, head trauma, stroke, CNS hypoxia, cerebral senility, multiinfarct dementia and other neurological conditions, as well as HIV and cardiovascular diseases, comprising administering an effective amount of a compound according to Formula (I).

In accordance with an embodiment of the invention there is provided a method of treating and/or preventing dementia in an Alzheimer's patient which comprises administering to the subject a therapeutically effective amount of a compound according to Formula (I) to inhibit the binding of an amyloid beta peptide (preferably, Aβ1-42) with nACh receptors, preferable α7nACh receptors, most preferably, human α7nACh receptors (as well as a method for treating and/or preventing other clinical manifestations of Alzheimer's disease that include, but are not limited to, cognitive and language deficits, apraxias, depression, delusions and other neuropsychiatric symptoms and signs, and movement and gait abnormalities).

The present invention also provides methods for treating other amyloidosis diseases, for example, hereditary cerebral angiopathy, normeuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis anthropathy, and Finnish and Iowa amyloidosis.

In addition, nicotinic receptors have been implicated as playing a role in the body's response to alcohol ingestion. Thus, agonists for α7nACh receptors can be used in the treatment of alcohol withdrawal and in anti-intoxication therapy. Thus, in accordance with an embodiment of the invention there is provided a method of treating a patient for alcohol withdrawal or treating a patient with anti-intoxication therapy comprising administering to the patient an effective amount of a compound according to Formula (I).

Agonists for the α7nACh receptor subtypes can also be used for neuroprotection against damage associated with strokes and ischemia and glutamate-induced excitotoxicity. Thus, in accordance with an embodiment of the invention there is provided a method of treating a patient to provide for neuroprotection against damage associated with strokes and ischemia and glutamate-induced excitotoxicity comprising administering to the patient an effective amount of a compound according to Formula (I).

Agonists for the α7nACh receptor subtypes can also be used in the treatment of nicotine addiction, inducing smoking cessation, treating pain, and treating jetlag, obesity, diabetes, sexual and fertility disorders (eg. Premature ejaculation or vaginal dryness, see U.S. Pat. No. 6,448,276), drug abuse (Solinas, Journal of Neuroscience (2007) 27(21), 5615-5620), and inflammation. Thus, in accordance with an embodiment of the invention there is provided a method of treating a patient suffering from nicotine addiction, pain, jetlag, obesity and/or diabetes, or a method of inducing smoking cessation in a patient comprising administering to the patient an effective amount of a compound according to Formula (I).

The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. Therefore, compounds with affinity for the α7nACh receptor on macrophages may be useful for human inflammatory diseases including rheumatoid arthritis, diabetes and sepsis. See, e.g., Czura, C J et al., J. Intern. Med., (2005) 257(2), 156-66, Wang, H. et al Nature (2003) 421: 384-388; de Jonge British Journal of Pharmacology (2007) 151, 915-929.

Thus, in accordance with an embodiment of the invention there is provided a method of treating a patient (e.g., a mammal, such as a human) suffering from an inflammatory disease, such as, but not limited to, rheumatoid arthritis, diabetes or sepsis, comprising administering to the patient an effective amount of a compound according to Formula (I).

The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol, Reproduct. 68: 1348-1353 2003). Consequently, selective α7 agents demonstrate utility for treating fertility disorders.

In addition, due to their affinity to α7nACh receptors, labeled derivatives of the compounds of Formula (I) (for example C11 or F18 labeled derivatives), can be used in neuroimaging of the receptors within, e.g., the brain. Thus, using such labeled agents in vivo imaging of the receptors can be performed using, for example PET imaging.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntingdon's disease, Pick's disease, Creutzfeldt-Jakob disease, HIV, cardiovascular disease, and head trauma as well as age-related cognitive decline.

Thus, in accordance with an embodiment of the invention there is provided a method of treating a patient suffering from, for example, mild cognitive impairment (MCI), vascular dementia (VaD), age-associated cognitive decline (AACD), amnesia associated w/open-heart-surgery, cardiac arrest, and/or general anesthesia, memory deficits from early exposure of anesthetic agents, sleep deprivation induced cognitive impairment, chronic fatigue syndrome, narcolepsy, AIDS-related dementia, epilepsy-related cognitive impairment, Down's syndrome, Alcoholism related dementia (Korsakoff Syndrome), drug/substance induced memory impairments, Dementia Puglistica (Boxer Syndrome), and animal dementia (e.g., dogs, cats, horses, etc.) comprising administering to the patient an effective amount of a compound according to Formula (I).

In accordance with an embodiment of the invention there is provided a method for improving or stabilizing cognitive function in a subject. In some embodiments, the method is for prevention or treatment of senile dementia, attention deficit disorders, Alzheimer's disease or schizophrenia.

In accordance with an embodiment of the invention there is provided a method of treating a central nervous system (CNS) disease or disorder. In some embodiments, a disease or disorder is selected from the group consisting of psychoses, anxiety, senile dementia, depression, epilepsy, obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, and pain.

Dosage of the compounds for use in therapy may vary depending upon, for example, the administration route, the nature and severity of the disease. In general, an acceptable pharmacological effect in humans may be obtained with daily dosages ranging from 0.01 to 200 mg/kg.

In some embodiments of the present invention, one or more compounds of formula (I) are administered in combination with one or more other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

For example, compounds of Formula (I), in forms as described herein, may be administered in combination with one or more other modulators of α7 nicotinic acetylcholine receptors. Alternatively or additionally, compounds of Formula (I), in forms as described herein, may be administered in combination with one or more other anti-psychotic agents, pain relievers, anti-inflammatories, or other pharmaceutically active agents.

Effective amounts of a wide range of other pharmaceutically active agents are well known to those skilled in the art. However, it is well within the skilled artisan's purview to determine the other pharmaceutically active agent's optimal effective amount range. The compound of Formula (I) and the other pharmaceutically active agent can act additively or, in some embodiments, synergistically. In some embodiments of the invention, where another pharmaceutically active agent is administered to an animal, the effective amount of the compound of Formula (I) is less than its effective amount would be where the other pharmaceutically active agent is not administered. In this case, without being bound by theory, it is believed that the compound of Formula (I) and the other pharmaceutically active agent act synergistically. In some cases, the patient in need of treatment is being treated with one or more other pharmaceutically active agents. In some cases, the patient in need of treatment is being treated with at least two other pharmaceutically active agents.

In some embodiments, the other pharmaceutically active agent is selected from the group consisting of one or more anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, β-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the compounds of Formula (I) include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the compounds of Formula (I) include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenyloin, primidone, or carbamazepine.

Cognitive enhancers that are used in combination with the compounds of Formula (I) include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5HT receptor modulators, 5HT_{1A} antagonists or agonists (including without limitation lecozotan and compounds described in U.S. Pat. Nos. 6,465,482, 6,127,357, 6,469,007, and 6,586,436, and in PCT Publication No. WO 97/03982) and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) IV inhibitors (e.g., roflumilast, arofylline, cilomilast, rolipram, RO-20-1724, theophylline, denbufylline, ARIFLO, ROFLUMILAST, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In some embodiments, the compound of Formula (I) and cognitive enhancer act additively or, in some embodiments, synergistically. In some embodiments, where a cognitive enhancer and a compound of Formula (I) of the invention are co-administered to an animal, the effective amount of the compound or pharmaceutically acceptable salt of the compound of the invention is less than its effective amount would be where the cognitive enhancer agent is not administered. In some embodiments, where a cognitive enhancer and a compound of Formula (I) are co-administered to an animal, the effective amount of the cognitive enhancer is less than its effective amount would be where the compound or pharmaceutically acceptable salt of the invention is not administered. In some embodiments, a cognitive enhancer and a compound of Formula (I) of the invention are co-administered to an animal in doses that are less than their effective amounts would be where they were no co-administered. In these cases, without being bound by theory, it is believed that the compound of Formula (I) and the cognitive enhancer act synergistically.

In some embodiments, the other pharmaceutically active agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In some embodiments, the compound of Formula (I) is administered together with another pharmaceutically active agent in a single administration or composition.

In some embodiments, a composition comprising an effective amount of the compound of Formula (I) and an effective amount of another pharmaceutically active agent within the same composition can be administered.

In another embodiment, a composition comprising an effective amount of the compound of Formula (I) and a separate composition comprising an effective amount of another pharmaceutically active agent can be concurrently administered. In another embodiment, an effective amount of the compound of Formula (I) is administered prior to or subsequent to administration of an effective amount of another pharmaceutically active agent. In this embodiment, the compound of Formula (I) is administered while the other pharmaceutically active agent exerts its therapeutic effect, or the other pharmaceutically active agent is administered while the compound of Formula (I) exerts its preventative or therapeutic effect.

Thus, in some embodiments, the invention provides a composition comprising an effective amount of the compound of Formula (I) of the present invention and a pharmaceutically acceptable carrier. In some embodiments, the composition further comprises a second pharmaceutically active agent.

In another embodiment, the composition further comprises a pharmaceutically active agent selected from the group consisting of one or more other antidepressants, anti-anxiety agents, anti-psychotic agents or cognitive enhancers. Antidepressants, anti-anxiety agents, anti-psychotic agents and cognitive enhancers suitable for use in the composition include the antidepressants, anti-anxiety agents, anti-psychotic agents and cognitive enhancers provided above.

In another embodiment, the pharmaceutically acceptable carrier is suitable for oral administration and the composition comprises an oral dosage form.

In some embodiments, one or more compounds of Formula (I) is administered in combination with antidepressant drug treatment, antipsychotic drug treatment, and/or anticonvulsant drug treatment.

In certain embodiments, a compound of Formula (I) is administered in combination with one or more selective serotonin reuptake inhibitors (SSRIs) (for example, fluoxetine, citalopram, escitalopram oxalate, fluvoxamine maleate, paroxetine, or sertraline), tricyclic antidepressants (for example, desipramine, amitriptyline, amoxipine, clomipramine, doxepin, imipramine, nortriptyline, protriptyline, trimipramine, dothiepin, butriptyline, iprindole, or lofepramine), aminoketone class compounds (for example, bupropion); in some embodiments, a compound of Formula (I) is administered in combination with a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenyloin, primidone, or carbamazepine. In some cases, the compound of Formula (I) is administered in combination with at least two drugs that are antidepressant drugs, antipsychotic drugs, anticonvulsant drugs, or a combination thereof.

### Pharmaceutical Compositions

In yet a further aspect, the invention refers to a pharmaceutical composition containing one or more compounds of Formula (I), in association with pharmaceutically acceptable carriers and excipients. The pharmaceutical compositions can be in the form of solid, semi-solid or liquid preparations, preferably in form of solutions, suspensions, powders, granules, tablets, capsules, syrups, suppositories, aerosols or controlled delivery systems. The compositions can be administered by a variety of routes, including oral, transdermal, subcutaneous, intravenous, intramuscular, rectal and intranasal, and are preferably formulated in unit dosage form, each dosage containing from about 1 to about 1000 mg, preferably from 1 to 600 mg of the active ingredient. The compounds of the invention can be in the form of free bases or as acid addition salts, preferably salts with pharmaceutically acceptable acids. The invention also includes separated isomers and diastereomers of compounds I, or mixtures thereof (e.g. racemic mixtures). The principles and methods for the preparation of pharmaceutical compositions are described for example in Remington's Pharmaceutical Science, Mack Publishing Company, Easton Pa.

When administered to an animal, one or more compounds of Formula (I), in any desirable form (e.g., salt form, crystal form, etc)., can be administered neat or as a component of a pharmaceutical composition that comprises a physiologically acceptable carrier or vehicle. Such a pharmaceutical composition of the invention can be prepared using standard methods, for example admixing the compound(s) and a physiologically acceptable carrier, excipient, or diluent. Admixing can be accomplished using methods well known for admixing a compound of Formula (I) and a physiologically acceptable carrier, excipient, or diluent.

Provided pharmaceutical compositions (i.e., comprising one or more compounds of Formula (I), in an appropriate form, can be administered orally. Alternatively or additionally, provided pharmaceutical compositions can be administered by any other convenient route, for example, parenterally (e.g., subcutaneously, intravenously, etc., by infusion or bolus injection, etc), by absorption through epithelial or mucocutaneous linings (e.g., oral, rectal, vaginal, and intestinal mucosa, etc.), etc. Administration can be systemic or local. Various known delivery systems, including, for example, encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound (and/or one or more metabolites thereof) into the bloodstream. The mode of administration may be left to the discretion of the practitioner.

In some embodiments, provided pharmaceutical compositions are administered orally; in some embodiments, provided pharmaceutical compositions are administered intravenously.

In some embodiments, it may be desirable to administer provided pharmaceutical compositions locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce a compound of Formula (I) into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound of Formula (I) can be formulated as a suppository, with traditional binders and excipients such as triglycerides.

In some embodiments, one or more compounds of Formula (I) can be delivered in a vesicle, in particular a liposome (see Langer, Science 249:1527-1533, 1990 and Treat et al., Liposomes in the Therapy of Infectious Disease and Cancer 317-327 and 353-365, 1989).

In some embodiments, one or more compounds of Formula (I) can be delivered in a controlled-release system or sustained-release system (see, e.g., Goodson, in Medical Applications of Controlled Release, vol. 2, pp. 115-138, 1984). Other controlled or sustained-release systems discussed in the review by Langer, Science 249:1527-1533, 1990 can be used. In some embodiments, a pump can be used (Langer, Science 249:1527-1533, 1990; Sefton, CRC Crit. Ref. Biomed. Eng. 14:201, 1987; Buchwald et al., Surgery 88:507, 1980; and Saudek et al., N. Engl. J. Med. 321:574, 1989). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61, 1983; Levy et al., Science 228:190, 1935; During et al., Ann. Neural. 25:351, 1989; and Howard et al., J. Neurosurg. 71:105, 1989).

As noted above, provided pharmaceutical compositions can optionally comprise a suitable amount of a physiologically acceptable excipient. Exemplary physiologically acceptable excipients can be liquids, such as water and oils, including those of petroleum, animal, vegetable, or synthetic origin, such as peanut oil, soybean oil, mineral oil, sesame oil and the like. For example, useful physiologically acceptable excipients can be saline, gum acacia, gelatin, starch paste, talc, keratin, colloidal silica, urea and the like. Alternatively or additionally, auxiliary, stabilizing, thickening, lubricating, and coloring agents can be used.

In some embodiments, a physiologically acceptable excipient that is sterile when administered to an animal is utilized. Such physiologically acceptable excipients are desirably stable under the conditions of manufacture and storage and will typically be preserved against the contaminating action of microorganisms. Water is a particularly useful excipient when a compound of Formula (I) is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Provided pharmaceutical compositions, if desired, can also contain minor amounts of wetting or emulsifying agents, or pH buffering agents.

Liquid carriers may be used in preparing solutions, suspensions, emulsions, syrups, and elixirs. A compound of Formula (I) can be dissolved or suspended in a pharmaceutically acceptable liquid carrier such as water, an organic solvent, a mixture of both, or pharmaceutically acceptable oils or fat. Such a liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Provided pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, pharmaceutical compositions in the form of a capsule are provided. Other examples of suitable physiologically acceptable excipients are described in Remington's Pharmaceutical Sciences 1447-1676 (Alfonso R. Gennaro, ed., 19th ed. 1995).

In some embodiments, a compound of Formula (I) (in an appropriate form) is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of one or more compounds of Formula (I) with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, provided pharmaceutical compositions can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some embodiments, the excipients are of pharmaceutical grade.

In some embodiments, one or more compounds of Formula (I) (in an appropriate form) can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where a compound of Formula (I) is to be administered by infusion, it can be dispensed, for example, with an infusion bottle containing sterile pharmaceutical grade water or saline. Where a compound of Formula (I) is administered by injection, an ampule of sterile water for injection or saline can be provided so that the ingredients can be mixed prior to administration.

In some embodiments, one or more compounds of Formula (I) (in an appropriate form) can be administered transdermally through the use of a transdermal patch. Transdermal administrations include administrations across the surface of the body and the inner linings of the bodily passages including epithelial and mucosal tissues. Such administrations can be carried out using the present in lotions, creams, foams, patches, suspensions, solutions, and suppositories (e.g., rectal or vaginal).

Transdermal administration can be accomplished through the use of a transdermal patch containing one or more compounds of Formula (I) (in an appropriate form) and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing a compound of Formula (I) with or without a carrier, or a matrix containing the active ingredient.

One or more compounds of Formula (I) (in an appropriate form) may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

One or more compounds of Formula (I) (in an appropriate form) can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In some embodiments a controlled- or sustained-release composition comprises a minimal amount of a compound of Formula (I) to treat or prevent one or more disorders, diseases or conditions associated with activity of α7 nicotinic acetylcholine receptors. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of one or more compounds of Formula (I) that promptly produces a desired therapeutic or prophylactic effect, and gradually and continually release other amounts of the compound to maintain this level of therapeutic or prophylactic effect over an extended period of time. To maintain a constant level of the compound a body, the compound can be released from the dosage form at a rate that will replace the amount of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, provided pharmaceutical compositions deliver an amount of a compound of Formula (I) that is effective in the treatment of one or more disorders, diseases, or conditions associated with activity (or inactivity) of α7 nicotinic acetylcholine receptors. According to the present invention, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. Effective dosage amounts described herein typically refer to total amounts administered; that is, if more than one compound of Formula (I) is administered, the effective dosage amounts correspond to the total amount administered.

The effective amount of a compound of Formula (I) for use as described herein will typically range from about 0.001 mg/kg to about 600 mg/kg of body weight per day, in some embodiments, from about 1 mg/kg to about 600 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In some embodiments, pharmaceutical compositions are provided in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. A unit dosage form can be, for example, a capsule or tablet itself, or it can be the appropriate number of any such compositions in package form. Such unit dosage form may contain, for example, from about 0.01 mg/kg to about 250 mg/kg, and may be given in a single dose or in two or more divided doses. Variations in the dosage will necessarily occur depending upon the species, weight and condition of the patient being treated and the patient's individual response to the medicament.

In some embodiments, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg;

A compound of Formula (I) can be assayed in vitro or in vivo for the desired therapeutic or prophylactic activity prior to use in humans. Animal model systems can be used to demonstrate safety and efficacy.

As depicted in the Exemplification below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### EXEMPLIFICATION

### Synthesis of Compounds

### Example 1

### 5-(4-Acetyl-[1,4]diazepan-1-yl)-pentanoic acid {5-[4-(tetrahydro-pyran-2-yloxy)-phenyl]-2H-pyrazol-3-yl}-amide

### Step a) 4-(Tetrahydro-pyran-2-yloxy)-benzoic acid methyl ester

4-Hydroxybenzoic acid methyl ester (4.0 g, 26.3 mmol) was mixed in acetone (20.0 mL), 3,4-Dihydro-2H-pyran (11.1 g, 131.5 mmol) and pyridinium p-toluene sulfonate (0.7 g, 2.6 mmol) were added and the reaction mixture stirred overnight at room temperature. The solvent was removed, the residue mixed in DCM (30 mL) and washed with NaOH (1 N). The organic phase was dried over Na₂SO₄ and concentrated. The product was obtained as a white solid (6.7 g, yield: 99%)

### C₁₃H₁₆O₄

Mass (calculated) [236]; (found) [M-THP+H⁺]=153.

LC Rt=3.69 min, 94% (10 min method)

¹H-NMR (dmso-d6): 1.40-1.85 (6H, m); 3.50-3.60 (2H, m); 3.65-3.75 (1H, m); 5.58 (1H, m); 7.10 (2H, d, J=8.9 Hz), 7.88 (2H, d, J=8.9 Hz).

### Step b) 3-Oxo-3-[4-(tetrahydro-pyran-2-yloxy)-phenyl]-propionitrile

To a solution of 4-(tetrahydro-pyran-2-yloxy)-benzoic acid methyl ester (6.7 g, 28.4 mmol) in dry toluene (30.0 mL) under N₂, NaH (50-60% dispersion in mineral oil, 2.3 g, 56.7 mmol) was carefully added. The mixture was heated at 80° C. and then dry CH₃CN was added dropwise (5.5 g, 133.9 mmol). The reaction was heated for 18 hours and the product precipitated from the reaction mixture as the sodium salt.

The reaction was then allowed to cool down to room temperature and the solid formed was filtered and then dissolved in water. The solution was then acidified with 2 N HCl solution, and at a pH of 4-5 the product was extracted with DCM.

The organic phase was dried over Na₂SO₄ and concentrated the product was used in the following step without further purification.

### Step c) 5-[4-(Tetrahydro-pyran-2-yloxy)-phenyl]-2H-pyrazol-3-ylamine

To a solution of 3-oxo-3-[4-(tetrahydro-pyran-2-yloxy)-phenyl]-propionitrile (4.1 g, 28.4 mmol), in absolute EtOH (30 mL) hydrazine monohydrate (1.65 mL, 34.0 mmol) was added and the reaction was heated at reflux for 4 hrs. The reaction mixture was allowed to cool to room temperature and the solvent was evaporated under reduced pressure. The orange residue was recrystallized from ethyl acetate and was obtained as a white solid (3.5 g, yield: 99%).

### C₁₄H₁₇N₃O₂

Mass (calculated) [259]; (found) [M+H⁺]=260, [M-THP+H⁺]=176.

LC Rt=1.93 min, 95% (10 min method)

¹H-NMR (DMSO-d6): 1.45-1.90 (6H, m); 3.50-3.56 (2H, m); 3.70-3.77 (1H, m); 5.45 (1H, m); 5.65 (1H, m), 7.00 (2H, d, J=8.8 Hz), 7.52 (2H, d, J=8.8 Hz).

### Step d) 5-(4-Acetyl-[1,4]diazepan-1-yl)-pentanoic acid {5-[4-(tetrahydro-pyran-2-yloxy)-phenyl]-2H-pyrazol-3-yl}-amide

### First Approach

A solution of 5-[4-(Tetrahydro-pyran-2-yloxy)-phenyl]-2H-pyrazol-3-ylamine (700 mg, 2.70 mmol) and diisopropylethylamine (697.8 mg, 5.40 mmol) in dry DMA (15 mL) was cooled to −10° C. (ice/water bath) under N₂; a solution of 5-bromovaleryl chloride (538.5 mg, 2.70 mmol) in dry DMA (5 mL) was added over 30 min. After 10 min at −10° C., completion of the reaction as monitored by LC-MS was generally observed. Acetylhomopiperazine (959.7 mg, 6.75 mmol) and NaI (404.6 mg, 2.70 mmol) were then added and the reaction heated at 40° C. overnight. The reaction was checked by HPLC-MS and 5-(4-Acetyl-[1,4]diazepan-1-yl)-pentanoic acid {5-[4-(tetrahydro-pyran-2-yloxy)-phenyl]-2H-pyrazol-3-yl}-amide was the main product, with 15% of 5-chloro-pentanoic acid {5-[4-(tetrahydro-pyran-2-yloxy)-phenyl]-2H-pyrazol-3-yl}-amide present as impurity. The solvent was evaporated and the product was purified by preparative HPLC. The title compound 5-(4-acetyl-[1,4]diazepan-1-yl)-pentanoic acid {5-[4-(tetrahydro-pyran-2-yloxy)-phenyl]-2H-pyrazol-3-yl}-amide was obtained contaminated with 10% of an unknown impurity (370 mg).

### Second Approach

A solution of 5-[4-(tetrahydro-pyran-2-yloxy)-phenyl]-2H-pyrazol-3-ylamine (1000 mg, 3.86 mmol) and diisopropylethylamine (997 mg, 7.71 mmol) in dry DMA (20 mL) was cooled to −10° C. (ice/water bath) under N₂; a solution of 5-bromovaleryl chloride (769 mg, 3.86 mmol) in dry DMA (5 mL) was added over 30 min. After 10 min at −10° C., completion of the reaction as monitored by LC-MS was generally observed. Acetylhomopiperazine (1371 mg, 9.64 mmol) and NaI (578 mg, 3.86 mmol) were then added and the reaction heated at 40° C. overnight. The reaction was checked by HPLC-MS and 5-(4-acetyl-[1,4]diazepan-1-yl)-pentanoic acid {5-[4-(tetrahydro-pyran-2-yloxy)-phenyl]-2H-pyrazol-3-yl}-amide was the main product, with 15% of 5-chloro-pentanoic acid {5-[4-(tetrahydro-pyran-2-yloxy)-phenyl]-2H-pyrazol-3-yl}-amide present as impurity. The solvent was evaporated and the reaction mixture treated with hot water. 5-chloro-pentanoic acid {5-[4-(tetrahydro-pyran-2-yloxy)-phenyl]-2H-pyrazol-3-yl}-amide precipitated as a white solid and was filtered off. The water phase was treated with saturated NaHCO₃ (10 mL) and the 5-(4-acetyl-[1,4]diazepan-1-yl)-pentanoic acid {5-[4-(tetrahydro-pyran-2-yloxy)-phenyl]-2H-pyrazol-3-yl}-amide obtained in the first approach was combined to this solution. The water phase was extracted with DCM and the product precipitated as a sticky white solid at the interphase. The product was recovered, treated with 2 N HCl and purified by preparative HPLC and converted to the HCl salt with HCl in Et₂O.

(531 mg, combined yield: 19%).

### C₂₁H₂₉N₅O₃

Mass (calculated) [399]; (found) [M+H⁺]=400.

LC Rt=double peaks 0.35 min, 1.12 min, 99% (10 min method)

¹H-NMR (DMSO-d6): 1.52-1.60 (2H, m); 1.63-1.72 (2H, m); 2.00 (3H, s); 2.02-2.11 (1H, m); 2.15-2.25 (1H, m), 2.28-2.38 (2H, m), 2.88-3.00 (1H, m), 3.02-3.12 (2H, m), 3.12-3.20 (1H, m), 3.30-3.60 (4H, m), 3.75-3.85 (1H, m), 3.95-4.05 (1H, m), 6.68 (1H, s), 6.79 (2H, d, J=8.7 Hz), 7.49 (2H, d, J=8.7 Hz), 10.44 (1H, s).

### Example 2

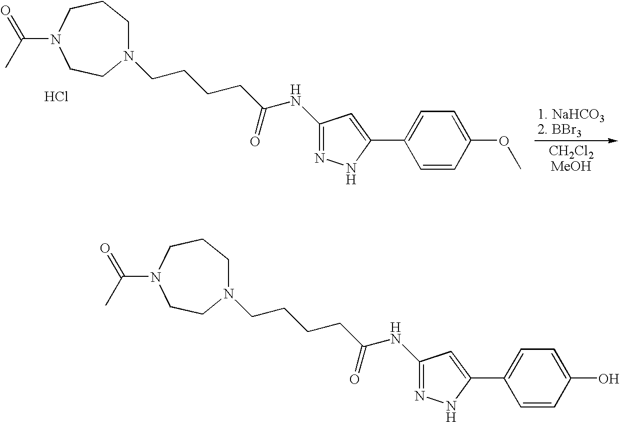

The HCl salt prepared in Example 1 (3.0 g, 6.6 mmol) was dissolved in 20 mL saturated sodium bicarbonate (pH 7 to 7.5). The resulting free base was extracted twice with 45 mL dichloromethane. The combined organic extracts was concentrated to ˜45 mL and cooled to 0-5° C. Excess boron tribromide reagent was added in portions (total 8.25 equiv.) and the reaction mixture stirred until reaction completion (<3% starting material by HPLC). The reaction mixture was quenched carefully with methanol and stripped to dryness to afford 2.7 g of the product (Compound I-1) as the dihydrobromide salt.

### Example 3

### Step a) 5-Bromo-pentanoic acid [5-(4-methoxy-phenyl)-1H-pyrazol-3-yl]-amide

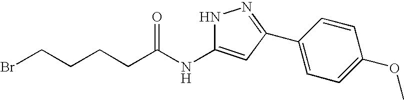

A solution of 5-bromovaleryl chloride (2.1 ml, 15.7 mmol, 1 eq) in dry DMA (35 ml) was cooled to −10° C. (ice/water bath) under N₂; a solution of 5-(4-Methoxy-phenyl)-1H-pyrazol-3-ylamine (3.0 g, 15.7 mmol, 1 eq) and diisopropylethylamine (2.74 ml, 15.7 mmol, 1 eq) in dry DMA (15 ml) was added over 30 min. After 2 hrs at −10° C., LC-MS analysis showed completion of the reaction (acylation on the pyrazole ring was also observed). The reaction was quenched by addition of H₂O (ca. 50 ml). Upon filtration from water and washing with Et₂O, 4.68 g of 5-bromo-pentanoic acid [5-(4-methoxy-phenyl)-1H-pyrazol-3-yl]-amide as a white powder were obtained (13.3 mmol, 85% yield).

mp=149.5-151.5° C.

C₁₅H₁₈BrN₃O₂ Mass (calculated) [352.23]; (found) [M+H⁺]=352.09/354.10

LC Rt=2.07, 95% (5 min method)

NMR (400 MHz, dmso-d6): 1.69-1.63 (2H, m); 1.81-1.75 (2H, m); 2.29 (2H, t); 3.52 (2H, t); 3.75 (3H, s); 6.75 (1H, bs); 6.96 (2H, d); 7.6 (2H, d); 10.28 (1H, s); 12.57 (1H, s).

### Step b) 5-[1,4]Diazepan-1-yl-pentanoic acid [5-(4-methoxy-phenyl)-2H-pyrazol-3-yl]-amide

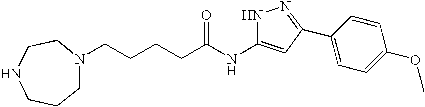

5-Bromo-pentanoic acid [5-(4-methoxy-phenyl)-1H-pyrazol-3-yl]-amide (0.211 g, 0.6 mmol) was dissolved in NMP (2 ml), sodium iodide (0.090 g, 0.6 mmol) was added followed by 1-Boc-homopiperazine (0.134 ml, 0.6 mmol) and diisopropylethylamine (0.105 ml, 0.6 mmol, 1 equiv). The reaction was stirred under N₂ at +50° C. for 18 hrs. Upon reaction completion (as monitored by LC-MS), the solvent was removed at reduced pressure and the resulting residue partitioned between H₂O/DCM (20 ml). The organic phase was evaporated and the crude purified by Si column (eluent DCM:MeOH 9:1 to DCM:MeOH NH₃ 2N 8:2), 0.027 g of the title compound as de-Boc (0.073 mmol, 12% yield) were recovered.

C₂₀H₂₉N₅O₂ Mass (calculated) [371.49]; (found) [M+H⁺]=372.30

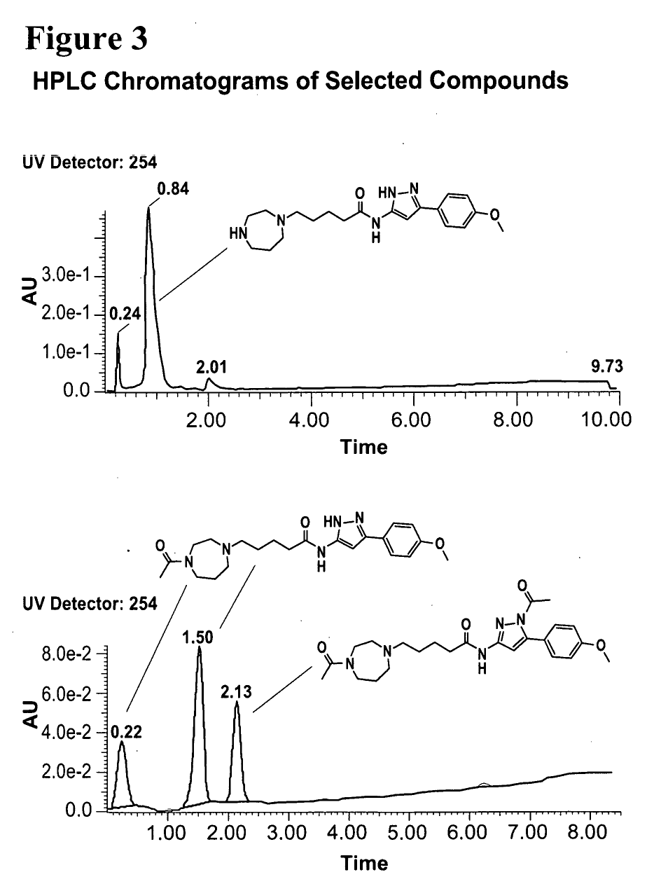

LC Rt=0.84, 99% (10 min method; see FIG. 3)

NMR (400 MHz, dmso-d6): 1.35-1.45 (2H, m); 1.50-1.60 (2H, m); 1.65-1.75 (2H, m); 2.25-2.30 (2H, m); 2.40-2.45 (2H, m); 2.55-2.65 (2H, m); 2.85-2.95 (4H, m); 3.77 (1H, s); 4.07 (1H, s); 6.75 (1H, s); 6.98 (2H, d, J=0.40 Hz); 7.61 (2H, d, J=0.40 Hz); 10.30 (1H, s); 12.60 (1H, s).

### Step c) 5-(4-Acetyl-[1,4]diazepan-1-yl)-pentanoic acid [5-(4-methoxy-phenyl)-2H-pyrazol-3-yl]-amide

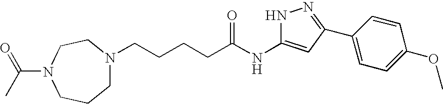

5-[1,4]Diazepan-1-yl-pentanoic acid [5-(4-methoxy-phenyl)-2H-pyrazol-3-yl]-amide (14.18 mg, 0.0382 mmol) was treated with 500 μL (3.60 μL, 0.0382 mmol of Ac₂O) of a solution of Ac₂O in DCE (solution prepared dissolving 36.0 μL of Ac₂O in 5.0 ml of DCE) and stirred overnight at RT. The reaction was monitored by LC-MS, the formation of about 30% of bisacetylated product was observed. No starting material was detected in the reaction mixture. The two products were separable by PrepHPLC (see FIG. 3).

The solvent was removed at reduced pressure and the resulting residue dissolved in 600 μL of a mixture H₂O/CH₃CN (75/25) and 200 μL of HCOOH and purified by prep HPLC. 6.0 mg of the title compound (0.0145 mmol, 34% yield) were recovered.

C₂₂H₃₁N₅O₃ Mass (calculated) [413.52]; (found) [M+H⁺]=414.46

LC Rt=0.22, 1.50, 99% (10 min method)

NMR (400 MHz, dmso-d6): 1.35-1.45 (2H, m); 1.49-1.58 (2H, m); 1.62-1.68 (1H, m); 1.70-1.78 (1H, m); 1.94-1.95 (3H, m); 2.24-2.30 (2H, m); 2.38-2.44 (2H, m); 2.50-2.56 (2H, m); 2.60-2.64 (1H, m); 3.38-3.45 (5H, m); 3.76 (3H, s); 6.72 (1H, s); 6.97 (2H, d, J=0.40 Hz); 7.61 (2H, d, J=0.40 Hz); 8.20 (1H, s); 10.33 (1H, s).

### Biological Activity

Cloning of Alpha7 Nicotinic Acetylcholine Receptor and Generation of Stable Recombinant Alpha 7 nAChR Expressing Cell Lines

Full length cDNAs encoding the alpha7 nicotinic acetylcholine receptor were cloned from a rat brain cDNA library using standard molecular biology techniques. Rat GH4C1 cells were then transfected with the rat receptor, cloned and analyzed for functional alpha7 nicotinic receptor expression employing a FLIPR assay to measure changes in intracellular calcium concentrations. Cell clones showing the highest calcium-mediated fluorescence signals upon agonist (nicotine) application were further subcloned and subsequently stained with Texas red-labelled α-bungarotoxin (BgTX) to analyse the level and homogeneity of alpha7 nicotinic acetylcholine receptor expression using confocal microscopy. Three cell lines were then expanded and one characterised pharmacologically (see Table 2 below) prior to its subsequent use for compound screening.

TABLE 2
Pharmacological characterisation of alpha7 nAChR stably
expressed in GH4C1 cells using the functional FLIPR assay

| Compound | EC₅₀ [microM] |
|---|---|
| Acetylcholine | 3.05 ± 0.08 (n = 4) |
| Choline | 24.22 ± 8.30 (n = 2) |
| Cytisine | 1.21 ± 0.13 (n = 5) |
| DMPP | 0.98 ± 0.47 (n = 6) |
| Epibatidine | 0.012 ± 0.002 (n = 7) |
| Nicotine | 1.03 ± 0.26 (n = 22) |

### Development of a Functional FLIPR Assay for Primary Screening

A robust functional FLIPR assay (Z′=0.68) employing the stable recombinant GH4C1 cell line was developed to screen the alpha7 nicotinic acetylcholine receptor. The FLIPR system allows the measurements of real time Ca²⁺-concentration changes in living cells using a Ca²⁺ sensitive fluorescence dye (such as Fluo4). This instrument enables the screening for agonists and antagonists for alpha 7 nAChR channels stably expressed in GH4C1 cells.

### Cell Culture

GH4C1 cells stably transfected with rat-alpha7-nAChR (see above) were used. These cells are poorly adherent and therefore pretreatment of flasks and plates with poly-D-lysine was carried out. Cells are grown in 150 cm² T-flasks, filled with 30 ml of medium at 37° C. and 5% CO₂.

### Data Analysis

EC₅₀ and IC₅₀ values were calculated using the IDBS XLfit4.1 software package employing a sigmoidal concentration-response (variable slope) equation:

Y=Bottom+((Top−Bottom)/(1+((EC₅₀/X)̂HillSlope))

### Assay Validation

The functional FLIPR assay was validated with the alpha7 nAChR agonists nicotine, cytisine, DMPP, epibatidine, choline and acetylcholine. Concentration-response curves were obtained in the concentration range from 0.001 to 30 microM. The obtained rank order of agonists is in agreement with published data (Quik et al., 1997, Mol. Pharmacol., 51, 499-506).

The assay was further validated with the specific alpha7 nAChR antagonist MLA (methyllycaconitine), which was used in the concentration range between 1 microM to 0.01 nM, together with a competing nicotine concentration of 10 microM. The IC₅₀ value was calculated as 1.31±0.43 nM in nine independent experiments.

### Development of Functional FLIPR Assays for Selectivity Testing

Functional FLIPR assays were developed in order to test the selectivity of compounds against the alpha1 (muscular) and alpha3 (ganglionic) nACh receptors and the structurally related 5-HT3 receptor. For determination of activity at alpha1 receptors natively expressed in the rhabdomyosarcoma derived TE 671 cell line an assay employing membrane potential sensitive dyes was used, whereas alpha3 selectivity was determined by a calcium-monitoring assays using the native SH-SY5Y cell line. In order to test selectivity against the 5-HT3 receptor, a recombinant cell line was constructed expressing the human 5-HT3A receptor in HEK 293 cells and a calcium-monitoring FLIPR assay employed.

### Screening of Compounds

Compounds of formula I, in appropriate forms, can be tested using the functional FLIPR primary screening assay employing the stable recombinant GH4C1 cell line expressing the α7 nAChR. Potency and selectivity (e.g., against the α1 nAChR, α3 nAChR and 5HT3 receptors) can be demonstrated. In some embodiments, an EC₅₀ within the range of about 1-2 nM is observed. The results of this assay using exemplary compounds of formula I can be seen in Table 3, below.

TABLE 3

| Compound | Alpha7 EC₅₀ |
|---|---|
| 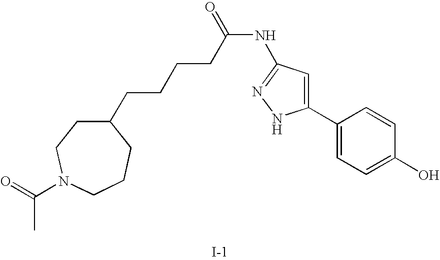 | 1.0 μM |
| 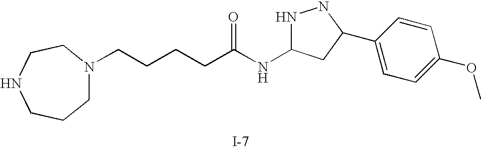 | 6.9 μM |

### Example 4

### Metabolite Profiles of Nicotine Alpha-7 Agonist Compound of Formula I in Rat and Dog Plasma

Rat plasma samples from single 6.7 mg/kg IV at 0, 10 min and 20.5 mg/kg IV at 0, 0.5, 4 hr post dosing; dog plasma samples from single 1.5 mg/kg IV at 0, 0.167, 1, 6 hr and 7.6 mg/kg PO at 0, 0.5, 2, 6 hr post dosing were analyzed by LC/MS. A standard of the parent compound was analyzed by LC/MS and there was no impurity detected.

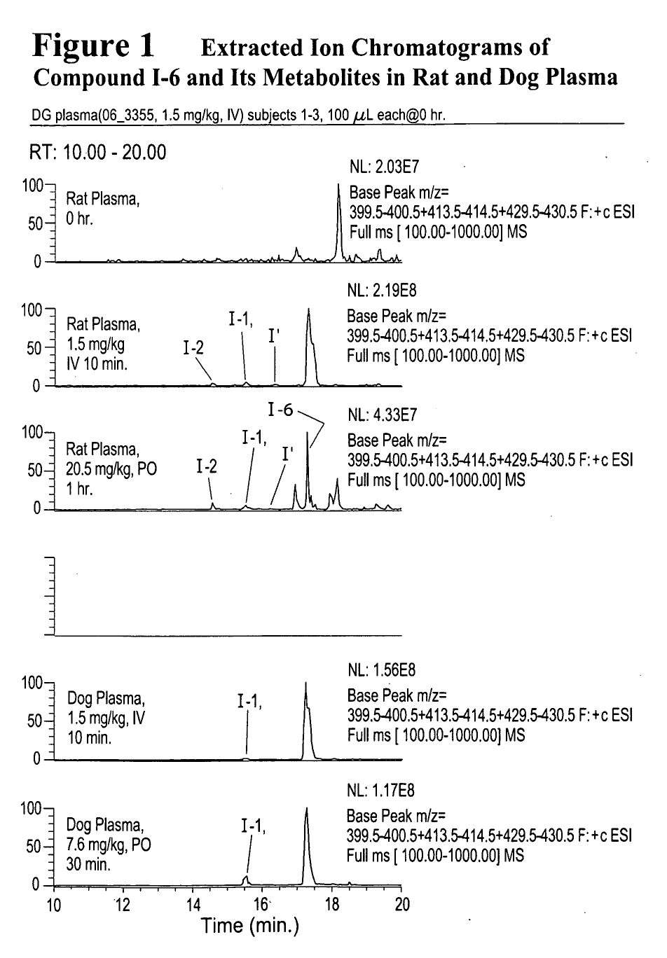

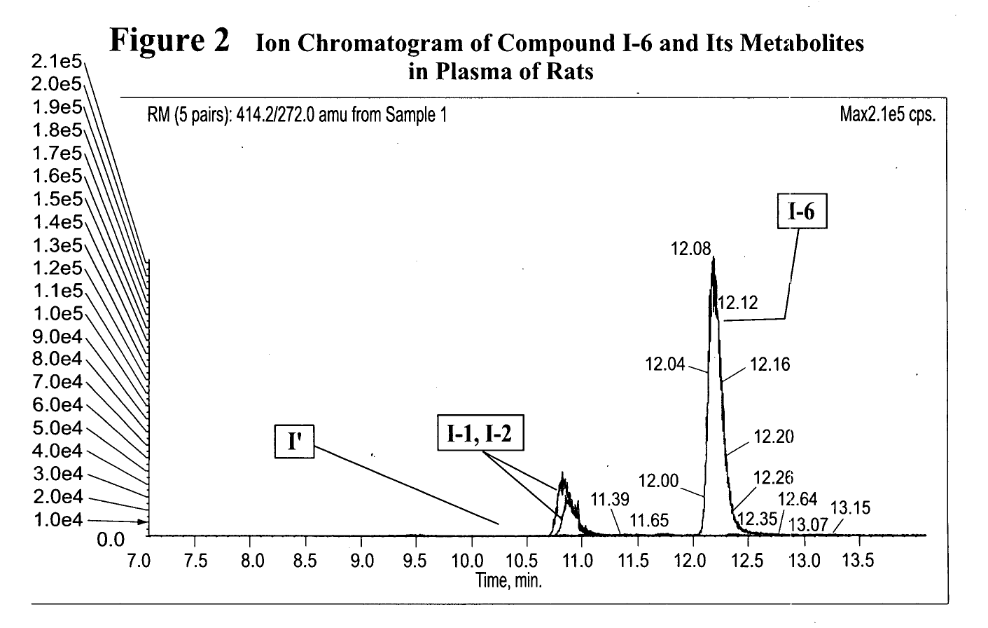

In rat plasma, a compound of formula I was the predominant compound-related material while metabolites were detected at very low levels. Mono-hydroxylated compound of formula I, O-desmethylated compound I-1 and its sulfate conjugate 1-2 were detected as metabolites. (See FIGS. 1 and 2; for compound I′ n=1, R¹=methyl, and R²═H)

In dog plasma, the parent compound was predominant while O-desmethylated compound I-1 was a very minor metabolite. (See FIG. 1).

1. A compound of formula I: 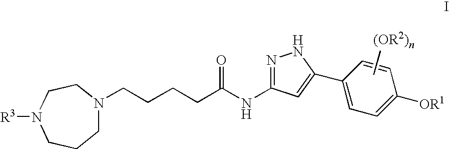
or a pharmaceutically acceptable salt thereof, wherein:
n is 0-4;
R¹ is hydrogen, methyl, or —SO₃H;
each R² is independently hydrogen or —SO₃H; and
R³ is hydrogen or —C(O)Me, provided that when R¹ is methyl and R³ is —C(O)Me, n is not 0.

2. A compound according to claim 1 selected from: 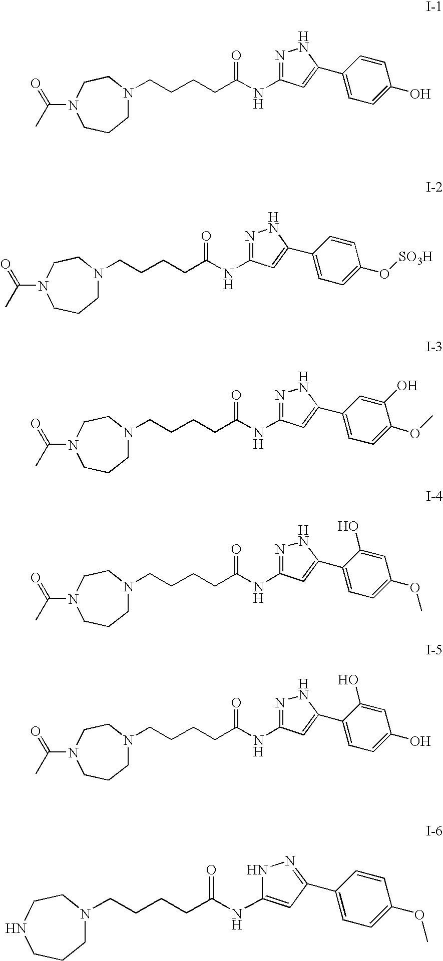
or a pharmaceutically acceptable salt thereof.

3. A compound according to claim 1, wherein n is 0-2.

4. A compound according to claim 3, wherein n is 1.

5. A compound according to claim 1, wherein R¹ is hydrogen.

6. A compound according to claim 1, wherein R¹ is methyl.

7. A compound according to claim 1, wherein R¹ is —SO₃H.

8. A compound according to claim 1, wherein R² is hydrogen.

9. A compound according to claim 1, wherein R² is —SO₃H.

10. A compound according to claim 1, wherein R³ is hydrogen.

11. A compound according to claim 1, wherein R³ is —C(O)Me.

12. A pharmaceutical composition containing a compound according to claim 1, with a pharmaceutically acceptable carrier or excipient.

13. A method for the treatment of neurological, neurodegenerative, psychiatric, cognitive, immunological, inflammatory, metabolic, addiction, nociceptive, and sexual disorders, comprising the step of administering to a subject in need thereof an effective amount of a compound according to any of claims 1 through 11 or a pharmaceutical composition of claim 12.

14. A method of treating a central nervous system (CNS) disease or disorder comprising administering to the subject a compound of any one of claims 1 through 11 or a pharmaceutical composition of claim 12.

15. The method of claim 14, wherein the disease or disorder is selected from the group consisting of psychoses, anxiety, senile dementia, depression, epilepsy, obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, and pain.

16. A method for improving or stabilizing cognitive function in a subject comprising administering to the subject a compound of any one of claims 1 through 11 or a pharmaceutical composition of claim 12.

17. A method for the prevention or treatment of diseases, conditions or dysfunctions involving the alpha 7 nAChR, which comprises administering to a subject in need thereof an effective amount of a compound according to any of claims 1 through 11 or a pharmaceutical composition of claim 12.

18. A method according to claim 17, for the prevention or treatment of senile dementia, attention deficit disorders, Alzheimer's disease and schizophrenia.
